(19) United States
(12) Patent Application Publication
(10) Pub. No.: US 2008/0233626 A1
(43) Pub. Date: Sep. 25, 2008

(54) Enhanced broad-spectrum UV radiation filters and methods

(76) Inventor: Yin-Xiong Li, Durham, NC (US)

Correspondence Address: CHARLES J. PRESCOTT, P.A., SUITE 115, 2033 WOOD STREET, SARASOTA, FL, 34237, US

(21) Appl. No.: 11/888,822

(22) Filed: Aug. 2, 2007

Related U.S. Application Data
(60) Provisional application No. 60/835,139, filed on Aug. 2, 2006.
(60) Provisional application No. 60/918,423, filed on Mar. 16, 2007.

(51) Int. Cl. C12N 13/00

(52) U.S. Cl. 435/173.1

(57) ABSTRACT

The present invention obtains from the discovery that combining traditional UV pigments, organic chemicals, or both, combined with NA, creates a broad spectrum UV absorbing additive that is much more efficient than using any of the ingredients by themselves. Methods for producing NA-coated particles as a UV protection additive to paints, fiberglass, plastic, polymers, siloxanes/silicates/reactive silanols, sealants or other film forming coatings or penetrating fluids and solid articles are contemplated in this invention as well as the coatings, sealants and other protectants and the coated and/or finished articles themselves.

### CROSS-REFERENCE TO RELATED APPLICATIONS

This application claims priority of U.S. Provisional Patent Application 60/835,139 filed Aug. 2, 2006 and U.S. Provisional Patent Application 60/918,423 filed Mar. 16, 2007.

### STATEMENT REGARDING FEDERALLY SPONSORED RESEARCH OR DEVELOPMENT

Not applicable

### INCORPORATION-BY-REFERENCE OF MATERIAL SUBMITTED ON A COMPACT DISC

Not applicable

### BACKGROUND OF THE INVENTION

1. Field of the Invention

This invention relates generally to the use of nucleic acid(NA)-containing materials, such as deoxyribonucleic acid and ribonucleic acid (collectively referred to as “NA”) which protect genetic material of living organisms from environmental hazards. More particularly, this disclosure relates to combining NA with other materials with UV-absorbing or blocking properties or network-forming properties as enhanced broad-spectrum ultraviolet radiation filters; the interposition of a barrier in the form of a liquid, semi-solid, or solid barrier that contains NA plus UV absorbing material between a source of UV radiation and a living organism; the protection of articles from UV damage by coating, impregnating, or otherwise interposing a barrier that contains a nucleic acid plus UV absorbing material between a source of UV radiation and the article; methods for combining NA and UV-absorbing or blocking chemicals, solid particles or pigments, particularly metal oxides, other network-forming organic molecules such as fatty acids, amino acids, and yeast extracts to produce enhanced UV-filter additives; methods for coating particles, particularly nanoparticles of metal oxides and oxidates, with NA; methods for producing liquid, semi-solid, or solid UV barriers; and articles with inherent UV-filtering or protection properties by being coated, impregnated, or otherwise treated with a combination of NA with particles, particularly nanoparticles of metal oxides and oxidates, chemicals known to absorb or block UV, other network-forming organic chemicals, or a blend.

2. Description of Related Art

Metal oxide pigments, particularly titanium dioxide and zinc oxide, physically block (reflect) UV radiation; a variety of organic chemicals including para-aminobenzoic acid (PABA) and esters thereof, benzophenones, and cinnemates absorb UV radiation, most notably in the UVB range (290-320 nm). More recently, U.S. Pat. No. 6,117,846 (incorporated herein by reference) disclosed that nucleic acid-containing materials, such as deoxyribonucleic acid and ribonucleic acid, their polymers and derivatives (hereafter referred inclusively as nucleic acids or (NA)) are excellent ultraviolet radiation filters, especially by absorbing genetic hazard ultraviolet radiation. Lyles, in U.S. Pat. No. 6,890,912 teaches a narrower version of the Li '846 patent by disclosing use of DNA of very large size of at least 10,000 base pairs.

The following is excerpted from an article titled “Relationship Between Ozone Depletion and Variations and Increased Skin Cancer” written by applicant and published for medical device 510K application.
Ozone depletion and variations correlate with the 5% yearly increases in skin cancer. It has raised the concern of very short wavelength ultraviolet-B radiation (UV-B, 280-320 nm) being a major contributor for the increasing skin cancer rates. These short wavelength UV photons with high energy have high affinity to attack the genetic information carrier molecule, DeoxyNucleic Acid (DNA). It causes multiple types of molecular damages of DNA. The most severe clinical consequence of these damages is skin cancer. The extent of DNA damage increases logarithmically as the UV-B wavelength decreases due to an innate affinity of the DNA molecule that absorbs these short-wavelength/high-energy UV photons. For live forms on earth, the necessity to evolve protection in the short wavelength UV-B has been previously mitigated by the filtering properties of the atmospheric ozone layer.
The causes of increases UV radiation in wavelengths below 300 nm can be attributed to both manmade and natural causes. However, it should be noted that for either reason, the effect is to create an “environmentally induced carcinogen” by increasing dangerous short wavelength UV-B. In mid- and high latitudes, ozone depletion is significant and can be related to increased skin cancer. In central latitudes, although ozone is somewhat depleted, most scientists agree that the nominally 5% to 10% historically depleted levels that were reached are now rebounding. However, what is little known and unpredictable is that, with the current state of the atmosphere, natural weather variations caused by frontal, cyclonic and volcanic activity can cause ozone variations far greater than ozone depletion. However, scientists also have come to the sobering realization that manmade events such as the explosion of a rogue nuclear weapon can duplicate the natural variation effects and could cause even larger ozone variations.
Prior to significant ozone depletion that began in the late twentieth century, very little UV-B with a wavelength below 300 nm has reached the Earth's surface and anything in the 290 nm range was considered inconsequential. In addition, even with today's technology, measurement of radiation in the 290- to 280 nm range is not reliable due to detector sensitivity and low photon flux and, as such, very little has been published on the levels.
The effects of high-energy, low-wavelength UV-B are being documented. The depletion of stratospheric ozone results in increased UV (ultraviolet) light below 300 nm and has significant effects on biological systems. Photons with a wavelength of 300 nm or less are so powerful that scientists have shown exposure to UVR of these wavelengths cause human beings and other organisms to be susceptible to such things as mutagenesis, carcinogenesis and cell death. These biological consequences occur because DNA is damaged. The extent of DNA damage increases logarithmically as the UV-B wavelength decreases due to an innate affinity of DNA molecule that absorbs these high-energy wavelengths. This is why UV sterilization lamps, which generate UV at 254 nm, are so effective in killing organisms. A lesser-known fact is that erythermal action spectrum flattens out and remains constant while DNA damage curves accelerate logarithmically at wavelengths below 300 nm distinguish DNA damage from erythema.
Worldwide, UVR is the most common mutagen that people are exposed to in their daily lives. It either causes changes in the DNA of genes by interfering with the genetic coding system or it causes direct chromosome damage and it inhibits the function of our naturally occurring DNA repair mechanisms. Currently, among all forms of UV-induced damage, the problem that is most pressing problem to humans is the DNA damage that UV causes in skin cells. DNA damage is clearly the pathological foundation for mutagenesis, carcinogenesis, and skin aging.
Protection systems for 300 nm and longer wavelengths are available. The sun protection factor (SPF) rating that is used to quantify the relative degree of protection offered by commercial sunscreen products is based upon the erythemal standard, a biological format and morphological criteria unjudged to the ability to provide protection against DNA damage caused by UVR, particularly in the portion of the UVR spectrum below 300 nm. Current commercial sunscreens offer protection against erythema at 300 nm and above.
There are no practical and comprehensive protective aids or medical devices currently available that offer protection at higher energy UV wavelengths below 300 nm, where a much smaller dosage can cause the same apoptotic or mutagenic effect as much larger doses of UV at wavelengths greater than 300 nm. The appended paper, “Results of Combining Nucleic Acids with Marine Collagen Fibers to Produce a Composite Ultraviolet Filter,” details a proposed medical device to protect skin from the low-wavelength UV-B, and also from UV-C wavelengths that may be encountered from UV lamps or at high-altitude exposure.

It would be of great benefit to provide a natural genetic UV radiation filter that can selectively and specifically block UV radiation that induces DNA damage. The medical device that is herein proposed is based on a concept wherein modified nucleic acids are used to selectively filter nucleic acid damaging UVR that can cause harm to plants, animals, and humans. It has been tested on and measured against a novel standard, known as genetic protection factor, or GPF.

### BRIEF SUMMARY OF THE INVENTION

Combining traditional UV pigments, organic chemicals, or both, with NA creates a broad spectrum UV absorbing additive that is more efficient than using any of the ingredients by themselves. NA strands appear to form a network that links with certain particles and chemicals at regular intervals along the strand. Because the organic NA network is soft, the NA-additive complex is easily smoothed out to form a uniform film with a uniform dispersion of other additives along the strands that more efficiently absorbs UV radiation than if either the NA or the particles or the UV-absorbing chemicals were simply dispersed in a liquid, suspension, or gel. It also is believed that a similar effect is obtained by combining NA, particles, UV-absorbing chemicals, or a blend with other network-forming organic molecules such as yeast extract, amino acids, or fatty acids. UV absorption efficiency at frequencies across the entire range from UVA through UVC are recorded and presented.

The present invention obtains from the discovery that combining traditional UV pigments, organic chemicals, or both, combined with NA, creates a broad spectrum UV absorbing additive that is much more efficient than using any of the ingredients by themselves. Methods for producing NA-coated particles as a UV protection additive to paints, fiberglass, plastic, polymers, siloxanes/silicates/reactive silanols, sealants or other film forming coatings or penetrating fluids and solid articles are contemplated in this invention as well as the coatings, sealants and other protectants and the coated and/or finished articles themselves.

### EXAMPLE 1

An example embodiment includes adding NA and zinc oxide particles, preferably as NA-coated nanoparticles, to an otherwise inert emulsion containing network-forming collagen “fibers” marketed by Englehard Corporation as MICROPATCH. The resulting compound may be applied topically to interpose a UV-absorbing/blocking barrier between the skin of humans or animals and a natural or artificial source of UV radiation. The quantity of NA-coated particles, the size of the particles, and addition of other UV-absorbing chemicals may be adjusted to filter biologically significant UV radiation from UVA through UVC. The level of MICROPATCH additive, other network forming organic chemicals, and otherwise inert ingredients can be adjusted to impart various levels of resistance to moisture and longevity. Because the NA absorbs UV and then releases the absorbed energy as heat without being destroyed, UV protection is afforded the wearer theoretically until the NA is washed or scrubbed off.

### OTHER EXAMPLES

Another advantage of NA-coated nanoparticles contemplated in this invention is the ability to suspend such particles in low viscosity fluids. Another advantage of NA-coated nanoparticles contemplated is the ability to produce clear, transparent, or translucent barrier creams, emulsions, or coatings. Particle types and preferred concentration ranges include, but are not limited to, 1-20% zinc oxide, 1-20% titanium dioxide, iron oxide, zirconium oxide and/or cerium oxide.

### Methods of Use and Production

Non-limiting methods for producing fluids, lotions, emulsions, and creams, coated plastic and polymers, and coated fibers and cloth were disclosed in U.S. Pat. No. 6,117,846. Similar methods apply to this invention with regard to producing liquids, gels, emulsions, and coated solid materials with NA in combination with particles, UV-absorbing chemicals, network-forming molecules, or a blend. Example articles containing or coated with NA and applications thereof of were disclosed in U.S. Pat. No. 6,117,846 and apply to the current invention.

Non-limiting methods for producing NA-coated particles as a UV protection additive to paints, fiberglass, plastic, polymers, siloxanes/silicates/reactive silanols, sealants or other film forming coatings or penetrating fluids and solid articles are contemplated in this invention, as well as the coatings, sealants, and other protectants and the coated and/or finished articles themselves. For example, a preferred embodiment includes NA-coated zinc oxide nanoparticles added to a silane blend which is subsequently hydrolyzed with other additives to produce a silanol sol that may be applied as a coating to produce a clear, thin coating that mitigates UV damage of the coated article. A similar coating may be applied to optical lenses, windows, or UV lamp bulbs to filter out genetically damaging UV radiation for the life of the coating. A similar coating or sealant may be applied to colored canvas, cloth, paint, or wood to impart fading resistance due to exposure to UV radiation.

The following embodiments and aspects thereof are described and illustrated in conjunction with systems, tools and methods which are meant to be exemplary and illustrative and not limiting in scope. In various embodiments one or more of the above-described problems have been reduced or eliminated while other embodiments are directed to other improvements. In addition to the exemplary aspects and embodiments described above, further aspects and embodiments will become apparent by reference of the drawings and by study of the following descriptions.

### BRIEF DESCRIPTION OF THE DRAWINGS

FIG. 1 illustrates transmittance of 1%-NA/Collagen fiber composite product with different dilutions measured between UV wavelength 220-325 nm.

FIG. 1A illustrates transmittance of 1%-NA/Collagen fiber composite product with different dilutions measured between UV wavelength 325-400 nm.

FIG. 2 illustrates transmittance of four chosen UV wavelength points for various dilutions of a 1%-NA/Collagen fiber composite product.

FIG. 3 illustrates scatter plot transmittance versus dilution for some representative discreet UV wavelengths.

FIG. 4 illustrates typical agar plates after irradiation of plasmid DNA, transfection into E. Coli, plate inoculation with E. Coli and Ampicillin and incubation for 24 hours.

FIG. 5 illustrates the relative average colony counts for barrier-free plasmid DNA/E. Coli with plastic wrap only and with NA/fiber composite barrier at high and low dose UVB.

FIG. 6 illustrates the relative effects of irradiation intensity and barriers on UV-induced damage to Plasmid DNA.

FIG. 7 illustrates the nucleic acid embedded filters blocking different wavelengths of UV energy.

FIG. 8 illustrates the formation of marine collagen macro-co-polymer and resulted nano-structure image under electronic scanning microscopy.

### DETAILED DESCRIPTION OF THE INVENTION

### Logical Tree for the Central Concept

The central concept of this disclosure includes a macro-copolymer network which filters physical and chemical hazard factors to protect genetic material in animal, human and object surfaces. The physical and chemical factors include UV, high-energy radiation (α, β and gamma rays), and neural, cell and DNA poison chemicals such as smoking derivative components and other DNA, RNA high affinity binding components.

The composition includes a macro-copolymer network that has highly ordered three-dimensional organizations at the nanometer level. Their conformation preference provides appropriate sites to host small molecules in divalent ions (e.g. Ca2+, Zn2+, Ba2+, SR2+among others), electromagnetic radiation shielding nanoparticals that include lead oxide, copperized lead, boron 10, boron nitride, boron carbide, polyethylene/boron, metal oxide/carbon, aluminum, Lithium, Yttrium, Zirconium, Titanium, lithium hydride, uranium and superparamagnetic iron oxide and other organic components. The macro-copolymer network can be formed by naturally existed bio-molecules including carbohydrates, proteins, lipids and nucleic acids as well as other organic molecules such as siloxanes, silicates, reactive silanols, sealants, polyethylene, carbon filaments and fiber-polymers.

The carbohydrates include alginates, agarose, sucrose, cellulose and resin. The proteins or peptides includes collages, yeast extracts, tryptone, elastin, as well as vegetable and marine micropatches. The lipids or fatty acid include C20-40 acid, polyethylene and Performacid 350 acid, as well as vitamins and retinoic acid. The nucleic acids include natural or synthetic DNA, RNA (size range from 1-5000 bp, single or double strands), polydeoxyribonucleic acids, polyribonucleic acids, as well as adenine, thymine, cytosine, guanine and their modified derivatives such as poly-thymine or dithymine.

The small components include oxidate pigment, such as zinc oxide, titanium, dioxide, iron oxide and cerium oxide, amino acid. The formed macro-copolymer network is generated from a single type of molecule or in combinations of molecules. The formed macro-copolymer network can be physical forms that include nano-confirmation structure, cream, lotion and gel, as well as liquid, semi-solid or solid states.

The formed macro-copolymer network absorbs DNA damaging UV particles in short wavelength with high energy from 220-300 nm generated from natural or artificial resources. The formed macro-copolymer network also absorbs DNA damaging UV particles in wavelength from 300-400 nm from natural or artificial resources.

The formed macro-copolymer network protects DNA damage and gene mutation in vivo and in vitro. The in vivo protection is applied to animals as well as humans. The in vitro protection is applied on material for UV filtering of fibers, papers, metals, glass and any surface.

### General Test Procedures

Procedures based on U.S. Pat. No. 6,117,846 were employed to produce an additive consisting of nucleic acids (NA) which have been found to exhibit outstanding ability to absorb biologically harmful ultraviolet (UV) radiation and convert the absorbed energy into heat. The NA additive was added to a typical skin cream base containing marine collagen fibers. Successive dilutions of the NA/fiber composite were irradiated with UV light and UV transmittance through the composite were plotted versus wavelength. These measurements were compared to transmittance measurements through distilled water (control) and through an FDA-cleared UV-blocking macro-fiber cloth (K920240). Additional measurements were made using plasmid DNA and bacterial transformation assay to measure the biological effects of unfiltered UV and filtered with the NA/fiber composite.

The completed study indicates the NA/fiber composite blocked approximately 99% of UV radiation (220-305 nm wavelengths) which was comparable to the FDA-cleared macro-fiber cloth. Dilutions up to 300 fold increased transmittance only to 1-2% at 251-252 nm. Observations from a transformation assay show that UV wavelengths 220-325 nm damaged all plasmid DNA at 0.15 J/cm² irradiation dose and approximately 80% of the plasmid DNA at 0.015 J/cm². Filtering UV through the NA/fiber composite increased survival rates to over 46% and 90%, respectively. An accelerated method that relates physical measurements of UV transmittance to biological damage is proposed.

### Prototype NA/Fiber Composite Creams

The procedure for producing the NA additive was as follows: (1) Double-stranded DNA from salmon milt (OD260/OD280 ratio between 1.5-2.0) was autoclaved at 121° C. for 30 minutes and then (2) filtered through a 0.2 μm filter for sterilization. Then, (3) the double-stranded DNA was denatured into single-stranded DNA by incubating the DNA at 98-100° C. for 5 minutes, followed by (4) immediately dipping the solution into ice water. (5) The solution of single-stranded DNA was then brought to a concentration of 5% DNA by weight (W/V) in TE buffer (10 mM Tris.Cl, pH 7.0, EDTA 1 mM). (6) One volume of this DNA solution was then mixed with 4 volumes of a solution consisting of 50% by weight 1-cyclohexy-3-(2-morpholinoethyl) carbodiimide metho-p-toluene solfonate (CMC) in 0.2 M sodium 2-(N-morpholino) ethanesulfonate, at pH 6.0. (7) The concentrated NA was attached to micro-fine particles using a proprietary process. Two stock solutions were prepared by mixing the NA additive with distilled water as follows:

| | |
|---|---|
| Stock solution A | 5.8% w/carrier (2.5 gram in 43 ml) |
| Stock solution B | 2.91% w/carrier (1.25 gram in 43 ml) |

A.I.G. Technologies (AIG) prepared a translucent cream base (clear when applied to the skin) using ingredients typically found in skin care products. AIG then added 12.5 ounces (5% of final sample) of 1% marine collagen fiber solution (Englehard Moisturizing Marine Micropatch® Composition Sheet #1 dated Apr. 25, 2006). From this base, 43 ml of the each stock solution was blended to make two, 250 ml samples of NA/fiber composite creams of approximately 1% and 0.5% NA additive, respectively.

### Spectrophotometry

Transmittance values through diluted samples were measured using a Beckman Model DU-65 spectrophotometer. The device produced plots of transmittance vs. wavelength at 1-nm intervals for two separate ranges:
Part I tests: 220-325 nm
Part II tests: 325-400 nm
   Optical density (OD) is the absorbance of an optical element for a given wavelength λ per unit distance

$${OD}_{λ} = \frac{{A}_{λ}}{l} = - \frac{1}{l} {log}_{10} T = \frac{1}{l} {log}_{10} ( \frac{{I}_{0}}{I} )$$

### Where:

I=the distance that light travels through the sample (i.e., the sample thickness), measured in cm

A_{λ}=the absorbance at wavelength λ

T=the per-unit transmittance

I₀=the intensity of the incident light beam

I=the intensity of the transmitted light beam

Transmittance: In optics and spectroscopy, transmittance is the fraction of incident light at a specified wavelength that passes through a sample.

$$T = \frac{I}{{I}_{0}}$$

where I₀ is the intensity of the incident light and I is the intensity of the light coming out of the sample. The transmittance of a sample is usually given as a percentage, defined as

$$T \% = \frac{I}{{I}_{0}} * 100 \%$$

Transmittance is related to absorbance A as

$$A = - {log}_{10} T = - {log}_{10} \frac{I}{{I}_{0}} or A = 2 - log T \%$$

where T% is the percent transmittance and T is “per one” transmittance. Note that the term transmission refers to the physical process of light passing through a sample, whereas transmittance refers to the mathematical quantity.

Samples were diluted to achieve optical densities (OD) in the range of 0.1-3.0 OD, where OD=1 means that 50% of visible light is transmitted through the sample. Samples were prepared by adding a 10-40 mg cream sample to an eppendorf tube, adding 1000 ml of distilled water, and blending for 1-3 minutes using a Vortex-genie 2 (Scientific Industries). Higher dilution folds or ratios were made through successive dilutions. 50 μl of solution was transferred by pipette into a sample curette which was then placed into the spectrophotometer for measurements. Measurements plots were generated for each UV range. Repeat measurements were made on different samples representing a specific dilution. The Beckman device was calibrated and normalized to a curette containing distilled water set to OD=0 (100% transmittance). A water measurement was made at the end of all test runs (Parts I-III) as a final calibration check.

FDA-cleared UV-blocking fabric samples were cut from a white shade scarf purchased from Sun Precautions, Inc. Samples were cut to fit and be placed in the UV light path, behind the sample curette. 50 μl of distilled water was added to the curette and transmittance was measured. Three different samples were measured for comparison with transmittance through diluted 1% NA/fiber composite cream samples, distilled water controls, and water-fiber curettes.

### Alternate Test Methods

Plasmid DNA UV Exposure and E. coli Transformation Assay Procedure

A method for evaluating the biological protection that various materials provide was devised based on the procedures disclosed in U.S. Pat. No. 6,117,846. Biological effects measurements were used to determine if these effects could be correlated with the spectrophotometer results. Appendix A herebelow details the methods employed to:
prepare plasmid DNA that produces enzymes that impart ampicillin-resistance to E. coli bacteria,
create sets of plasmid DNA that are either unexposed, UV-exposed, or UV-exposed with interposing barrier,
transfect the target E. coli bacteria with the plasmid DNA,
inoculate transformed E. coli onto ampicillin-containing agar plates, and,
incubation of the agar plates at 37° C. for 24 hours and count resulting colonies.
   96-cell tissue culture plates containing plasmid DNA were irradiated in a UV-stratalinker 2400 (Stratagen, La Jolla, Calif.) having a 4000 microwatt capacity with peak energy at nominally 254 nm. Irradiated plasmid DNA transformed E. coli colonies were counted and compared with non-irradiated plasmid transformed E. coli plates prepared at the same time (control).

### Measurements and Observations

### Spectrophotometry

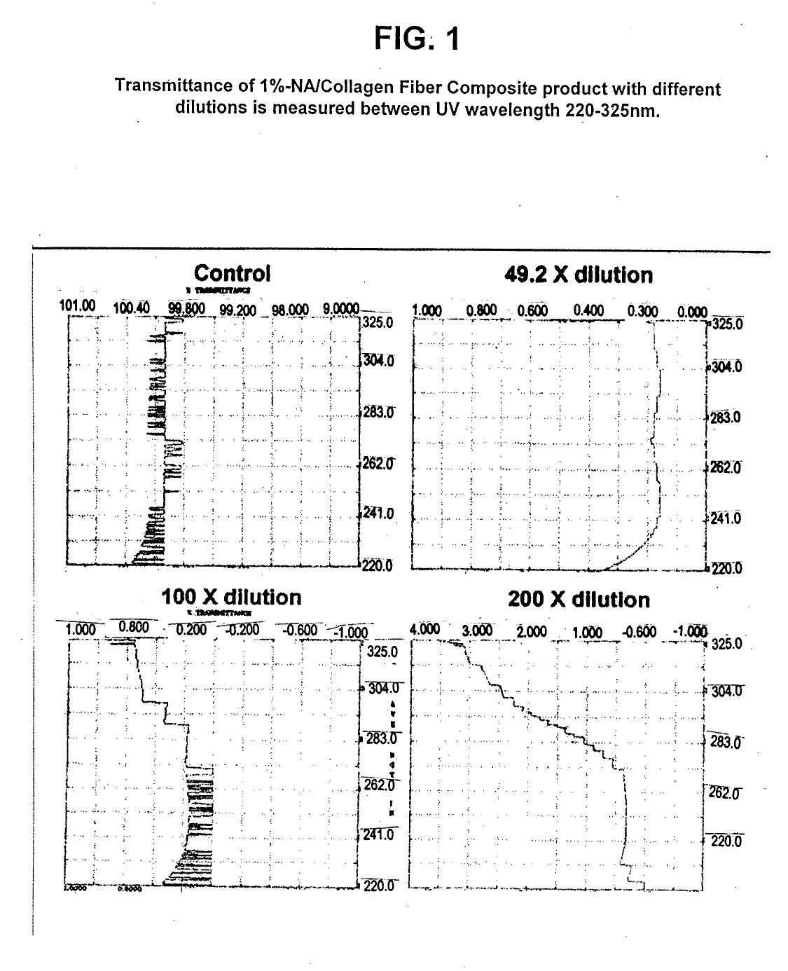

Example Beckman spectrophotometer output plots for the range of 220-325 nm were produced for a variety of NA/fiber composite cream samples diluted with water (expressed as dilution folds) to achieve measurable optical density (OD). In most cases, at least two different samples representing the same dilution were plotted. FIG. 1 presents examples of original data that illustrate some of the UV spectrum of transmittances of four tested samples with different dilutions in 200-325 nm. FIG. 1A presents additional examples of original data which shows the UV spectrum of transmittances of four tested samples with different dilutions in 325-400 nm.

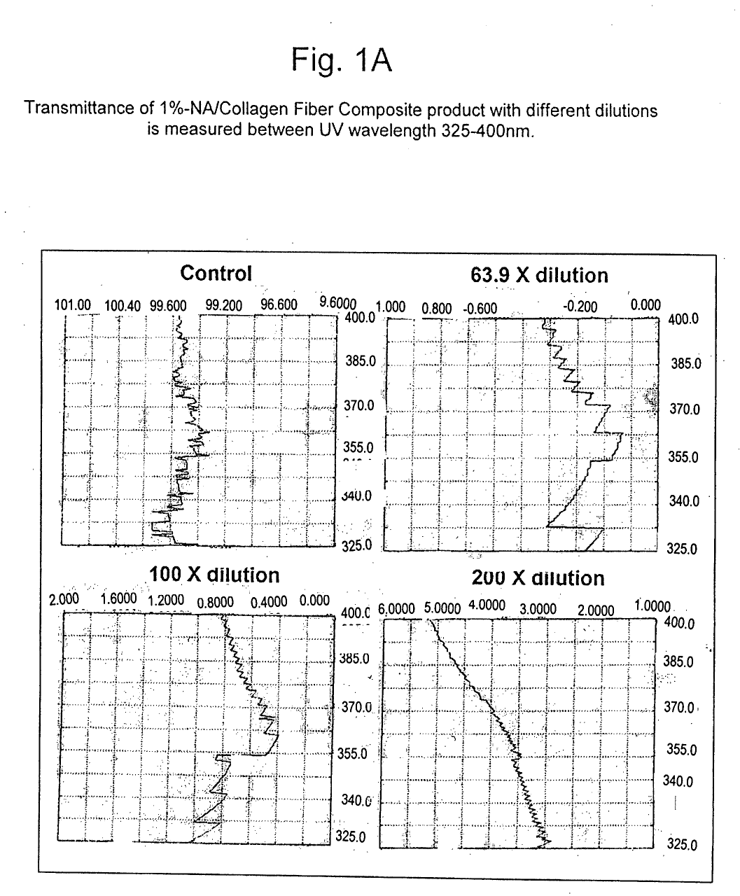

To display and compare results, each plot was visually examined and transmittances vs. wavelength data-pairs were measured from the original plot graphs. Table I presents four points of the whole measured wavelength region (220-305 nm) and depicts the manual data extracted from the Beckman plots. FIG. 2 summarizes the data of the transmittances of four chosen points in the 220-305 nm region.

TABLE 1
Transmittance at Some Discrete UV Wavelengths for Various
Dilutions of a 1%-NA/Collagen Fiber Composite Cream

| | | | Measured Transmittance (Percent) at | | |
|---|---|---|---|---|---|
| Sample | Dilution | Test Run | Representative Wavelengths | | |
| Number | Fold | Number | 220 | 251.5 | 304 |
| Water | Part I | | 100.3 | 99.9 | 101 |
| | Control | | | | |
| Water | Part II | | 100.3 | 99.8 | 101 |
| | Control | | | | |
| Water | End of all | | 108 | 102.4 | 102 |
| | tests | | | | |
| Fiber 1 | | | 0 | 0.18 | 0.18 |
| Fiber 2 | | | 0.35 | 0.32 | 0.33 |
| Fiber 3 | | | 0.35 | 0.32 | 0.33 |
| Averages | | | 0.23 | 0.27 | 0.28 |
| Standard | | | 0.20 | 0.08 | 0.09 |
| Deviation | | | | | |
| 1A | 100 | 1 | 0.3 | 0.18 | 0.3 |
| | | 2 | 0.3 | 0.18 | 0.5 |
| 2A | 1000 | 1 | 9 | 18 | 38 |
| | | 2 | 9 | 18 | 37 |
| | | 3 | 9 | 18 | 37 |
| | | Average | 9 | 18 | 37.3 |
| 3A | 10000 | 1 | 68 | 84 | 93 |
| | | 2 | 68 | 85 | 93 |
| | | Average | 68 | 84.5 | 93 |
| 4a | 100000 | 1 | 80 | 98 | 100 |
| | | 2 | 81 | 99 | 100 |
| | | Average | 80.5 | 98.5 | 100 |
| 1Aa | 200 | 1 | 0 | 0.035 | 2.45 |
| | | 2 | 0 | 0.036 | 2.45 |
| 1Ab | 400 | 1 | 0.07 | 2.2 | 10.8 |
| | | 2 | 0.08 | 2.1 | 10.8 |
| 1Ac | 800 | 1 | 6.5 | 14 | 32.6 |
| | | 2 | 6.5 | 14.3 | 32.6 |
| A1 | 36.7 | | | | |
| A1a | 73.4 | | 0.35 | 0.18 | 0.34 |
| A1b | 164.8 | | 0.31 | 0.16 | 1.48 |
| A1c | 293.6 | | 0.7 | 1.3 | 7.5 |
| A1d | 587.2 | | | | |
| A2 | 22.6 | | | | |
| A2a | 45.2 | | 0.3 | 0.18 | 0.19 |
| A2b | 90.4 | | 0.35 | 0.17 | 0.5 |
| A2c | 180.8 | | 0.3 | 0.3 | 2.5 |
| A2d | 361.6 | | | | |
| A3 | 24.6 | | | | |
| A3a | 49.2 | | 0.35 | 0.16 | 0.17 |
| A3b | 98.3 | | 0.35 | 0.16 | 0.5 |
| A3c | 196.7 | | 0.35 | 0.34 | 2.3 |
| A3d | | | | | |
| A4 | 63.9 | 1 | 0 | 0.19 | 0.18 |
| A4a | 127 | 1 | 0.5 | 02 | 1.6 |
| A4b | 255.5 | 1 | 0.7 | 1.2 | 6.2 |
| A4c | 511.4 | 1 | 5.1 | 9.6 | 22 |
| A4d | 1022.4 | 1 | 22.8 | 27.8 | 45.5 |

Recording three significant figures implies greater accuracy than can be accorded the data in Table 1. Each data-pair includes the error associated with the actual vs. calculated dilution of the sample, the error associated with manually reading both a wavelength value (y axis) and a transmittance value (x-axis) from the Beckman plot, and the variation error in measurements between and within each run of the Beckman spectrophotometer. Despite the accumulation of these errors, data-pairs exhibited acceptable consistency. Bar charts correlating all the data depicted in FIG. 2 suggest relative consistency among the multiple Beckman plots over the entire range of dilutions.

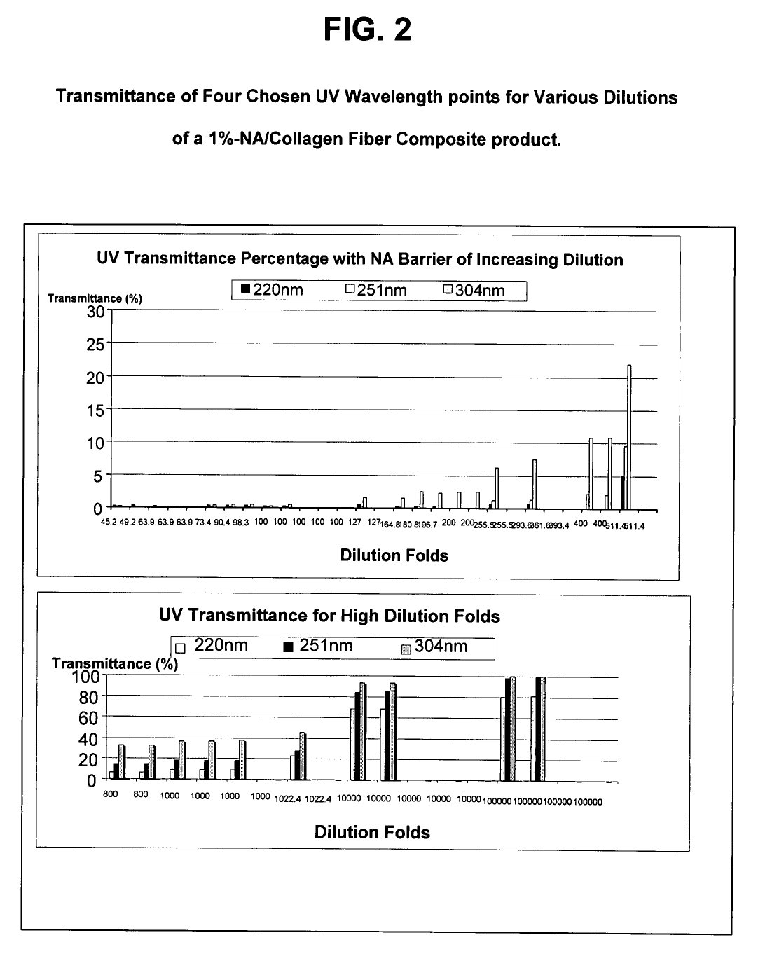

Measured UV transmittance for samples exceeding 500 fold dilution were considered unacceptably high. A scatter plot of data from 45-511 dilution folds was generated using MICROSOFT® EXCEL software and is depicted in FIG. 3. The software was also used to generate trend lines for 251 nm and 355 nm wavelengths.

Plasmid DNA UV Exposure and E. coli Transformation Assay Procedure

Agar plates representing irradiated and non-irradiated plasmid DNA/E. coli inoculants were prepared per Appendix A. Only plasmid DNA protected from or free from damaging UV radiation will be capable of transfecting E. coli with the ability to produce ampicillin-resistant enzyme. Accordingly, the number of transfected E. coli colonies existing on ampicillin-contained agar plates will be inversely proportional to the amount of genetically damaging UV absorbed by the plasmid DNA.

It was found that the UV-blocking capacity of the plastic film and of the tissue culture plates masked effects of the UV-absorbing NA/fiber composite cream solutions. The UVB dose-intensity was increased from 0.015 J/cm2 to 0.15 J/cm2. Cultures were exposed to these doses for approximately one minute. The higher dose was sufficient to damage the plasmid DNA to where no E. coli colonies survived exposure to ampicillin. This dose represents about 1/20 of the annual average dose of American adults, though at a much higher intensity (approximately 500 times).

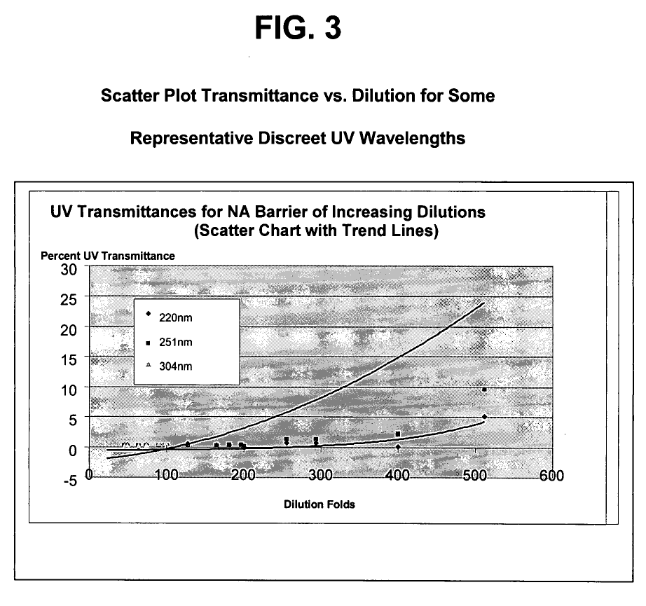

After a 24-hour incubation period, agar plates were examined and colonies were counted. A set of original experimental agar plate is imaged in FIG. 4. Table 2 collates colony counts for various test runs.

TABLE 2
Colony Counts for Agar Plates after Irradiation of Plasmid
DNA, Transfection with E. Coli, Plate Inoculation with E. Coli
and Ampicillin, and Incubation for 24 Hours

| | Plate | Plate | Plate | Plate | Plate | Plate |
|---|---|---|---|---|---|---|
| | 1 | 2 | 3 | 4 | 5 | 6 |
| UV 254 nm | 0 | 0.15 | 0.015 | 0.15 | 0.15 | 0.015 |
| J/cm2 | | | | | | |
| Sara Plastic | None | None | None | Yes | Yes | Yes |
| film | | | | | | |
| Sara + NA/ | None | None | None | None | Yes | Yes |
| fiber | | | | | | |
| composite | | | | | | |
| Experiment 1a | 108 | 1 | 18 | 0 | 56 | 98 |
| Experiment 1b | 89 | 1 | 19 | 1 | 57 | 94 |
| Experiment 2a | 94 | 1 | 19 | 1 | 58 | 89 |
| Experiment 2b | 90 | 1 | 20 | 2 | 59 | 91 |
| Average | 95.25 | 1 | 19 | 1 | 57.5 | 93 |
| SE | 4.39 | 0 | 0.41 | 0.41 | 0.65 | 1.96 |
| % | 100.000% | 1.0% | 19.9% | 1.0% | 60.4% | 97.6% |
| SE | 4.6% | 0.0% | 0.4% | 0.4% | 0.7% | 2.1% |

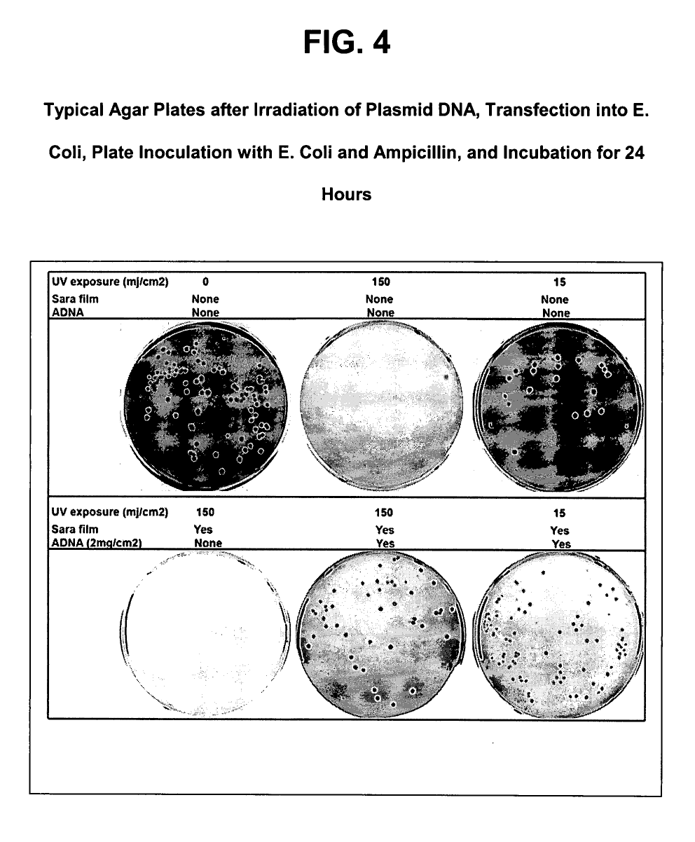

FIG. 5 depicts the data of Table 2. Using Plate 1 as the control, FIG. 5 reveals that a low UVB dose (0.015 J/cm2-Plate 3) allows approximately 20% of plasmid DNA to function whereas the high UVB dose (0.15 J/cm2) destroys virtually all of the plasmid DNA, regardless of whether any plastic wrap is present (Plates 2 and 4). Adding the 1% NA/fiber composite barrier applied at 2 mg/cm2 increases protection from nil to 60% at high intensity/high dose UVB (Plate 5). Increasing the dilution fold of barrier cream used on a separate test plate produced the same protection rate (60%) as was seen for the undiluted cream. Increasing the concentration of plasmid DNA irradiated was checked to determine test sensitivity. Increasing the concentration of plasmid DNA by a factor of five increased relative efficiencies at all irradiation levels and barriers.

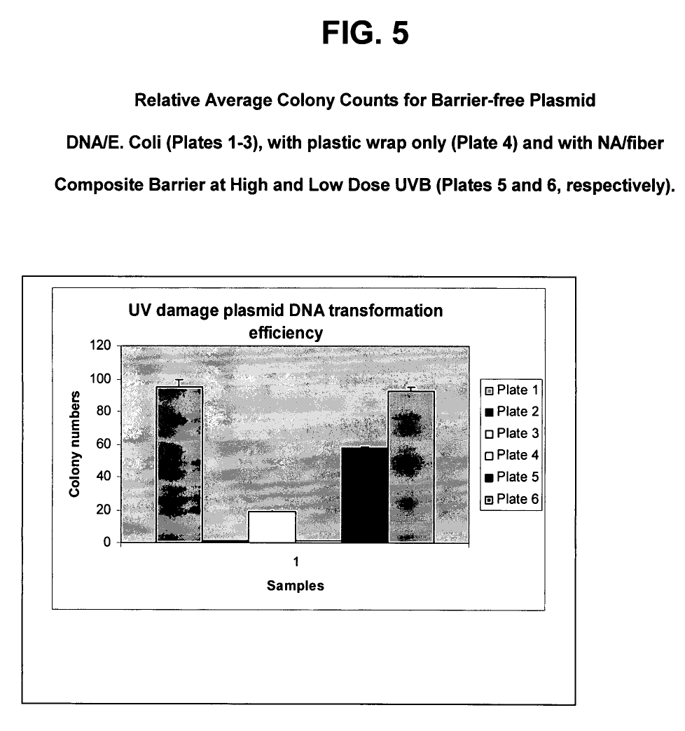

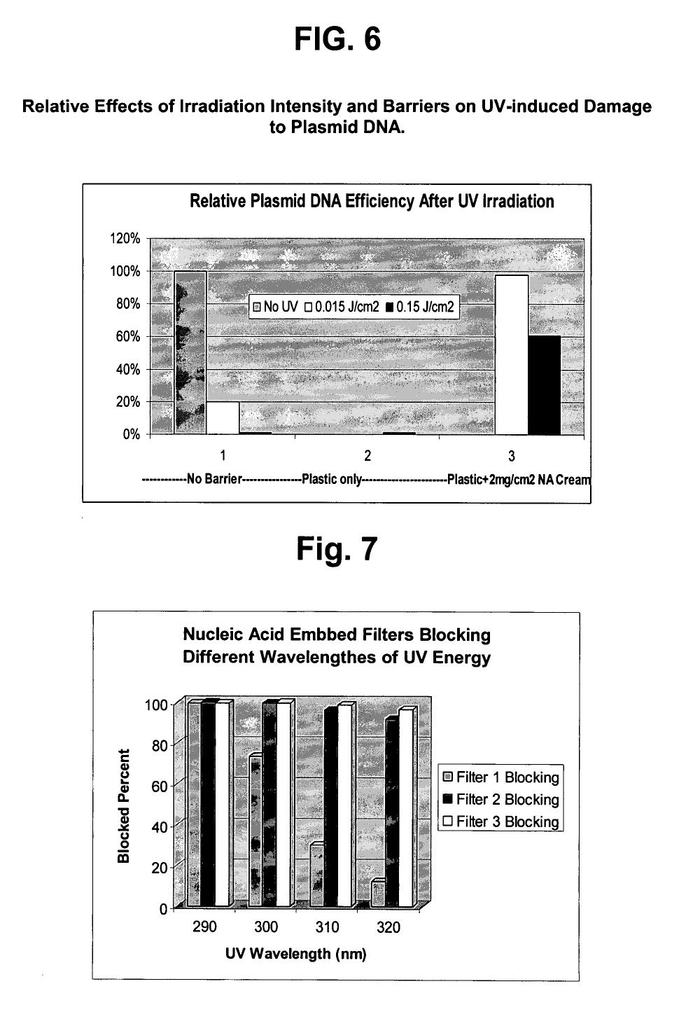

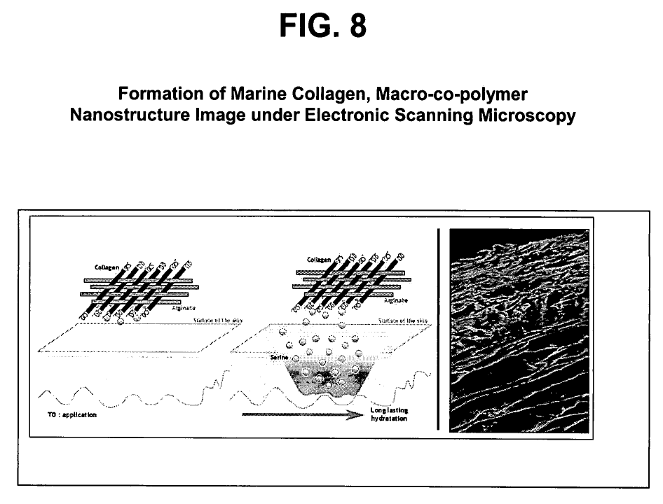

Comparing Plates 1 and 6 in FIG. 5 indicates that the 1% NA/fiber composite barrier applied at 2 mg/cm2 protected 98% of the plasmid DNA from low dose UVB, or five times the protection than with no clear barrier at all (Plate 3). The effects of interposing the 1% NA/fiber composite barrier on the relative plasmid DNA transformation efficiency at high and low UV irradiation intensities is depicted in FIG. 6.

### Summary of Findings

1. The UV barrier effects of nucleic acids (NA) and derivatives of NA disclosed in U.S. Pat. No. 6,117,846 were validated by spectrophotometer measurements and biological effects measurements.
2. The feasibility of combining NA with marine collagen fibers in a topical cream carrier was demonstrated.
3. The feasibility of using a plasmid DNA UV exposure and E. coli transformation assay analogous to biological effects measurements to validate physical transmittance measurements was demonstrated.
4. Even when the 1% NA/fiber composite cream was diluted from 64 to 300 fold, 98% of UVB transmittance was blocked.
5. These results are essentially equivalent to the amount of UV blocked by FDA-cleared macro-fabrics (labeled fiber1, fiber2, and fiber3 in Table 2).

### Discussion

The spectrophotometer tests successfully discriminated between low and high dilutions of the 1% NA/marine collagen fiber composite cream. As expected, reducing the density of UV-absorbing NA in the fluid increased the transmittance of UV at all wavelengths. At dilutions from 64 to 300 fold, 98% of UVB transmittance was blocked.

UV absorption from UVB, especially at biologically significant 254 nm wavelength was greater than 99% for dilutions up to 100 fold. This performance was the same level of protection provided by the FDA-cleared UV blocking fabric medical device.

The plasmid DNA UV exposure and E. coli transformation assay results validate the spectrophotometer results. The 1% NA/fiber composite cream at 2 mg/cm² over plastic wrap prevented 98% of plasmid DNA from becoming damaged at a 0.015 J/cm² dose of UVB (250 nm peak, 1 minute irradiation in the UV-stratalinker) compared with only 20% with no protection. Applying 10 times of this UV dose damaged all the plasmid DNA, unless the 1% NA/fiber composite cream was interposed as a protective barrier.

This method offers promise as an accelerated test for evaluating the effects of radiation on living organisms. The consistency of measurements shown in Table 2 indicates that the method produces repeatable results.

Increasing the intensity of UV (254 nm peak) increases DNA damage as expected. The barrier effects of plastic or plastic wrap can be overcome by sufficiently high intensity UVB, but may complicate estimation of the effects of interposed UV barrier materials. However, increasing the dilution fold (ratios) of barrier cream used on a separate test plate produced the same protection rate (60%) as was seen for the undiluted cream. Increasing the concentration of plasmid DNA irradiated was checked to determine test sensitivity. Increasing the concentration of plasmid DNA by a factor of five increased relative efficiencies at all irradiation levels and barriers. However, this may be because the higher concentration of plasmid DNA is shielding some of the plasmid in the center of the vial. While this validates the theory of the NA device, it suggests the concentration of plasmid DNA described in Appendix A is appropriate for screening tests.

Based on the extensive literature linking UV exposure of humans to premature aging, wrinkles, and skin cancer, use of UV protection is prudent. However, the FDA has cleared only one medical device based on macro-fabric barriers. Fabric barriers alone are impractical in some instances, and even when applied, do not protect uncovered portions of the body—particularly the face, hands, and eyes—from reflected UV. This disclosure demonstrates the feasibility of combining marine collagen micro-fibers with NA, as well as using a cream base to deliver the NA/fiber composite. Further, the NA/fiber composite was shown to provide UVB protection comparable to macro-fabrics cleared by the FDA to provide the same medical benefits.

Physical spectrophotometric measurements of barrier efficiency were validated by the Plasmid DNA UV Exposure and E. coli Transformation Assay Procedure. That is, physical measurements were analogous to biological effects measurements. It is reasonable to presume that the UV protection of organisms afforded by the 1% NA/fiber composite cream would be observed in in vivo tests.

### EXAMPLE 2

Agarose was tested as a second active UV absorbing agent with nucleic acid producing the results shown in Table 4. Nucleic acid was embedded into 1-2% agarose forming clear filters. The nucleic acid concentrations in Filter #1, #2, #3 and #4 are 0.0005%, 0.005%, 0.01% and 0.02% respectively. Table 4 is the summary of the measured data for blocking indoor tanning bed UV lamp CF26W, in UVB and UVA ranges as well as the UV energies (uW) required to produce erythema and melanogenesis.

TABLE 4

| | UVB (to | UVA (to | | |
|---|---|---|---|---|
| | 320 nm) | 400 nm) | Erythema | Melanogenesis |
| Filter #0 | 185.1 | 5030 | 35.3 | 102.9 |
| (without NA | | | | |
| coating) | | | | |
| Filter #1 | 135.3 | 4420 | 47.1 | 136.4 |
| Filter #2 | 33.2 | 2590 | 239.8 | 617.2 |
| Filter #3 | 26.1 | 1910 | 239.6 | 650.3 |
| Filter #4 | 0.6 | 550 | 2840.1 | 6375.1 |

### EXAMPLE 3

Table 3 presents the complete preferred formula which has been developed and ready for manufacture.

TABLE 3
UV Filtering Fabric

| | Trade Name | % w/w |
|---|---|---|
| A1 | Deionized Water | 59.225 |
| A2 | Plantaren 2000 | 0.500 |
| A3 | Glycerin | 1.000 |
| A4 | BioVera 200x Aloe Powder | 0.100 |
| A5 | Triethanolamine | 0.400 |
| A6 | Versene Na | 0.100 |
| B1 | Sensanov WR | 2.000 |
| B2 | Jeechem CTG | 3.000 |
| B3 | Permethyl 99A | 2.000 |
| B4 | Vitamin E Acetate | 0.250 |
| B5 | Crodamul SS | 2.000 |
| B6 | Polawax | 1.000 |
| B7 | DC 556 Fluid | 2.000 |
| C | Nucleic Acids | 1.000 |
| D | Zinc Oxide | 2.5 |
| E | Simulgel NS | 3.600 |
| F | Microcare MTG | 0.500 |
| G | Peppermint Oil AA-P2663 | 0.125 |
| | Marine Micropatch | 5.000 |

### Manufacturing Procedure

1. Into a suitable sized stainless steel kettle, capable of holding the entire batch and equipped with a sidesweep and lightening-type mixers, add ingredients A1-A6.
2. Heat to 70-75° C. Mix until uniform.
3. Into a suitable sized stainless steel kettle, equipped with a lightning type mixer, add ingredients B1-B7.
4. Heat to 70-75° C. Mix until uniform.
5. When both phases are uniform and at temperature, add Phase A into bottom of Phase B.
6. Mix until uniform.
7. Add ingredient C. Mix until uniform.
8. Homogenize until uniform.
9. Add ingredient D. Mix until completely dispersed.
10. Homogenize until uniform.
11. Begin to cool to 30° C. with city water.
12. At 40C, add ingredients E and f. Mix until uniform between additions.
13. At 35C, reduce mixer speeds and slowly add ingredient G.
14. Mix until uniform. Continue to mix until 30° C.

While a number of exemplary aspects and embodiments have been discussed above, those of skill in the art will recognize certain modifications, permeations and additions and subcombinations thereof. It is therefore intended that the following appended claims and claims hereinafter introduced are interpreted to include all such modifications, permeations, additions and subcombinations that are within their true spirit and scope.

### Appendix A

### Plasmid DNA UV Exposure and E. Coli Transformation Assay Procedure

Plasmid DNA pEGFP-N1 and PUC19 have been chosen as the target DNAs to be the biosensor for UV damage studies. pUC19 (GenBank/EMBL accession number L09137) is a commonly used E. coli cloning vector. It is a small, double-stranded DNA circle, 2686 base pairs in length, and has a high copy number. PUC19 expresses an ampicillin resistant gene in host cells. Plasmid DNA pEGFP-N1 pEGFP-N1 (Clontech. Catalog number 6085-1) encodes a red-shifted variant of wild-type GFP which has been optimized for brighter fluorescence and higher expression in mammalian cells (excitation maximum=488 nm; emission maximum=507 nm.). pEGFP-N1 encodes the GFPmut1 variant (4) which contains the double-amino-acid substitution of Phe-64 to Leu and Ser-65 to Thr.

The coding sequence of the EGFP gene contains more than 190 silent base changes which correspond to human codon-usage preferences. Sequences flanking EGFP have been converted to a Kozak consensus translation initiation site to further increase the translation efficiency in eukaryotic cells. The vector backbone also contains an SV40 origin for replication in mammalian cells expressing the SV40 T-antigen. A neomycin-resistance cassette (neo^{r}), consisting of the SV40 early promoter, the neomycin/kanamycin resistance gene of Tn5, and polyadenylation signals from the Herpes simplex thymidine kinase gene, allows stably transfected eukaryotic cells to be selected using G418. A bacterial promoter upstream of this cassette (Pₐₘₚ) expresses kanamycin resistance in E. coli.

Plasmid DNA, in concentrations of 50-100 ng/μl, is add to the 96-well tissue culture plate at 50 μl per well. The plate is placed directly into the UV stratalinker 2400 (Stratagen, La Jolla, Calif.) or is covered with Cloth Specimens that are coated—with or without nucleic acid. For liquid or semi-liquid targets, the entire plate is covered with a commercial plastic film (for example, Saranwrap) which is then coated with target sample to achieve a nominal coverage rate of 2 mg/cm2. The Plasmid DNA is irradiated by UV light in the Stratalinker 2400 at various UV energies, ranging from 125 to 150,000 μJ/m2. After the UV irradiation, the plasmid DNA will be diluted to 1 ng/μl concentration for transformation assay. The DH5 Chemically Competent E. coli (Catalog no. 18265-017, Invitrogen Life Technologies) has been chosen as the host cell.

Steps of this procedure are as follows:
1. Briefly centrifuge the plasmid DNA after UV exposure.
2. Remove one 500 μl tube of DH5 E. coli cells and thaw on wet ice.
3. Place the required number of sterile 1.5 ml microcentrifuge tubes on wet ice.
4. Gently mix cells with the pipette tip and aliquot 50 or 100 μl into each microcentrifuge tube.
5. Re-freeze any unused cells in the dry ice/ethanol bath for 5 minutes before returning the tube to the −70° C. freezer.
6. Pipet 1 μl (1 ng DNA) of each experiment DNA directly into the competent cells and mix by tapping gently. Do not mix by pipetting up and down. Store the remaining DNA samples at −20° C.
7. Incubate the vial on ice for 30 minutes.
8. Heat-shock for exactly 30 seconds in the 37° C. water bath for 50 μl volume (45 seconds for 100 μl transformation). Do not mix or shake.
9. Remove vial from the 37° C. bath and place on ice for 2 minutes.
10. Add 900 to 950 μl of pre-warmed S.O.B. medium (0.5% Yeast extract, 2.0% tryptone, 10 mM NaCl, 2.5 mM KCl, 10 mM MgCl₂, 10 mM MgSO₄) to each vial.
11. Place the vial in a microcentrifuge rack on its side and secure with tape to avoid loss of the vial. Shake the vial at 37° C. for exactly 1 hour at 225 rpm in a shaking incubator.
12. Spread 20 μl to 200 μl (prefer 100 μl) from each transformation vial on LB agar plate that contains 25 mg/ml kanamycin for pEGFP-N1 transformation, or 100 ug/ml ampicillin for pUC19 transformation.
13. Invert the plates and incubate at 37° C. overnight.
14. Count colony number of each plate and take images.

1. A method for protecting genetic material from genetic hazard electromagnetic/ultraviolet radiation-induced and cellular DNA attacking chemical induced damage, the method comprising:
placing a genetic hazard ultraviolet radiation-filtering barrier between a source of ultraviolet radiation and a target containing electromagnetic/ultraviolet radiation-sensitive genetic material, the barrier comprising an effective amount of an electromagnetic/ultraviolet radiation-filtering nucleic acid within a nucleic acid based polymer network and an active electromagnetic/ultraviolet absorbing and/or blocking material.

2. The method of claim 1, wherein said nucleic acid is taken from the group consisting of:
polydeoxyribonucleic acid, polyribonucleic acid, adenine, thymine, cytosine, guanine and derivatives and mixtures thereof.

3. The method of claim 1 wherein:
said active material includes UVA and/or UVB hydrophilic organic UV-screening agent, and/or a lipophilic organic UV-screening agent, and/or a mineral UV-screening nanopigment.

4. The method of claim 1, wherein said active material is taken from the group consisting of:
titanium oxide, iron oxide, zirconium dioxide, zinc oxide, cerium oxide, octyl methoxycinnamate, penzophenone-3, octocrylene, octyl salicylate, yeast extract, trypone, agarose, C20-40, sucrose, collagen, cellulose, elastin, retinoic acid, amino acid, polyethylene, perform acid 35D acid, L-serine, sodium alginate, algin, siloxane, silicates, reactive silanols and sealants.

5. The method of claim 1, wherein said active electromagnetic radiation blocking material is taken from the group consisting of:
lead oxide, copperized lead, boron 10, boron nitride, boron carbide, polyethylene/boron, metal oxide/carbon, aluminum, lithium, yttrium, zirconium, titanium, lithium hydride, uranium and superparamagnetic iron oxide.

6. The method of claim 1, wherein:
said polymer includes an ordered 3-dimensional network.

7. The method of claim 3, wherein:
said nucleic acid is present in an amount of up to about 20% (W/V).

8. The method of claim 1, wherein:
said nucleic acid is dilutible to a concentration of about 0.01% (W/V) while maintaining a UV transmittance of no greater than about 8%.

9. The method of claim 1, wherein:
said nucleic acid is dilutable to a concentration of about 0.015% (W/V) while maintaining a UV transmittance of no greater than about 3%.

10. The method of claim 1, wherein:
said nucleic acid is dilutable to a concentration of about 0.03% (W/V) while maintaining a UV transmittance of substantially all UV.

11. The method of claim 2 further comprising the steps of:
irradiating said coated or impregnated surface to induce cross-linking of the polydeoxyribonucleic acid or polyribonucleic acid to the polymer network.

12. A composition for protecting genetic material form genetic hazard ultraviolet radiation-induced damage, comprising:
an effective amount of an ultraviolet radiation-filtering nucleic acid within a nucleic acid based polymer network and an active UV absorbing and/or blocking material.

13. The composition of claim 12, wherein said nucleic acid is selected from the group consisting of:
polydeoxyribonucleic acid, polyribonucleic acid, adenine, thymine, cytosine, guanine and modified derivatives and mixtures thereof.

14. The composition of claim 12, wherein:
said active material includes UVA and/or UVB hydrophillic organic UV-screening agent, and/or a lipophylic organic UV-screening agent, and/or a mineral UV-screening nanopigment.

15. The composition of claim 12, wherein said active material is taken from the group consisting of:
titanium oxide, iron oxide, zirconium dioxide, zinc oxide, cerium oxide, octyl methoxycinnamate, penzophenone-3, octocrylene, octyl salicylate, yeast extract, trypone, agarose, C20-40, sucrose, cellulose, collagen, elastin, retinoic acid, amino acid, polyethylene, perform acid 35D acid, L-serine, sodium alginate, algin, siloxanes, silicates, reactive silanols, sealants.

16. The composition of claim 12, wherein:
said polymer includes an ordered 3-dimensional network.

17. The composition of claim 12, wherein:
said nucleic acid is present in an amount up to about 20% (W/V).

18. The composition of claim 12, wherein:
said nucleic acid is dilutible to a concentration of about 0.01% (W/V) while maintaining a UV transmittance of no greater than about 8%.

19. The composition of claim 12, wherein:
said nucleic acid is dilutable to a concentration of about 0.015% (W/V) while maintaining a UV transmittance of no greater than about 3%.

20. The composition of claim 12, wherein:
said nucleic acid is dilutable to a concentration of about 0.03% (W/V) while maintaining a UV transmittance of substantially all UV.

21. The composition of claim 12, further comprising the steps of:
irradiating said barrier to induce cross-linking of the polydeoxyribonucleic acid or polyribonucleic acid to the polymer network.
